(19) United States
(12) Patent Application Publication
(10) Pub. No.: US 2019/0221134 A1
(43) Pub. Date: Jul. 18, 2019

(54) Meal Management System

(71) Applicant: Life Log Technology, Inc., Tokyo (JP)

(72) Inventors: Shigeyuki TANAHASHI, Tokyo (JP); Kodai AMAN, Tokyo (JP)

(73) Assignee: Life Log Technology, Inc., Tokyo (JP)

(21) Appl. No.: 16/249,869

(22) Filed: Jan. 16, 2019

(30) Foreign Application Priority Data
Jan. 17, 2018 (JP) 2018-005497

(51) Int. Cl. G09B 19/00; G06K 9/00; G06K 9/62; G16H 20/60

(52) U.S. Cl. CPC G09B 19/0092; G06K 9/00664; G06K 2209/17; G16H 20/60; G06K 9/6202

(57) ABSTRACT

A meal management system is disclosed, which includes a user terminal in which an application program is to be executed and a server apparatus and managing a meal of a user. The application program causes a computer to transmit all the obtained meal images and the obtained images other than the obtained meal images to the server apparatus upon obtaining the meal images and the images other than the meal images, which have not yet been transmitted to the server apparatus. The server apparatus configured to receive the meal images and the images other than the meal images from the user terminal, extract the image feature amounts and the imaging dates and times of the meal images and the image other than the meal images, identify a meal item included in the meal image, based on a degree of matching between the image feature amount of the meal item registered in the meal item storage and the image feature amount of the meal image and the image other than the meal image, display a meal image analysis screen, and store meal management information based on the imaging date and time of the meal image and the meal item.

### RELATED APPLICATIONS

This present application is based upon and claims the benefit of priority of the prior Japanese Patent Application No. 2018-5497, filed on Jan. 17, 2018, the entire contents of which are incorporated herein by reference.

### FIELD

The disclosure is related to a meal management system.

### BACKGROUND

Conventionally, a meal management system and a diet management system that utilize calorie calculation and nutrition management based on ingested meal items (meal menu) to help health care and diet of a user are known. A user needs to input records such as meal item, meal date and time, meal type (breakfast, lunch, dinner) and the like.

Also, in recent years, when a user launches an application from a mobile terminal such as a smartphone and takes a meal photo with the attached camera, the meal item is automatically recognized by image analysis, so it is easy to record the meal item. There are also things that can be done.

As a technique related to this, for example, Patent Document 1 (Japanese Laid-open Patent Publication No. 2011-028382) discloses a meal element recording section for storing, for each user identifier, a recorded meal element image for each meal element in a meal ingested in the past and its nutritional component value; an ingested meal image receiving section for receiving an ingested meal image taken by the user; a meal element extraction section for extracting one or more meal element images from the ingested meal image; an image recognizing section for recognizing the most similar recorded meal element image, for each of the extracted meal element images, which are stored in the meal element recording section; a nutritional component value searching section for searching for a nutrient component value of the meal element corresponding to the detected recorded meal element image; a nutrition component value calculating section for totaling all the nutritional component values, and a nutrition component value recording section for accumulating and managing the nutrition component values for each user identifier.

Further, for example, Patent Document 2 (Japanese Laid-open Patent Publication No. 2007-122311) discloses a nutritional analysis apparatus which includes an image capturing section 1 for capturing an image of food, a food identifying section 2 for identifying a type of food from the captured image data, and an amount of meal from the captured image data; a food amount identifying section 3 nutritional analysis section 4 for carrying out a nutritional analysis from the food identifying section 2 and the food amount identifying section 3, and a nutrition analysis result display section 5 for displaying the nutrient analysis result, wherein the type of food is identified by the food identifying section 2, the amount of food is identified by the food amount identifying section 3 and the calorie nutrition analysis of the food is performed by the nutrition analysis section 4.

However, conventionally, in the case of managing the meal, it takes time and labor for the user to record each meal item every time the user eats. Furthermore, also in the case of the inventions of Patent Documents 1 and 2, it is necessary to activate the application, photograph a meal photo, and record the meal item each time the user takes a meal.

If the user forgets to record, it is also possible to collectively records while remembering past meals. However, it is difficult to accurately recall all the memories of the meal items eaten in the past, so it is highly likely that meal management itself will not continue for a long time due to inability to manage accurate meals, etc.

With respect to meal management and diet, proper continuous recording is the first step towards achievement. Many people try hard at first, but gradually stop recording because of the trouble. Therefore, in meal management applications and diet applications, how to reduce the hurdle of posting (recording) in order to increase the continuation rate is a subject.

### SUMMARY

According to one aspect, a meal management system for managing a meal of a user is provided, which includes a user terminal in which an application program is to be executed and a server apparatus, wherein
the application program causes a computer to
   obtain a meal image and an image other than the meal image, the meal image including information of date and time when the meal image was captured;
   store the meal images and the images other than the meal images in an image storage;
   obtain the meal images and the images other than the meal images, which have not been transmitted to the server apparatus, from the image storage at a timing when the application program is activated; and
   transmit all the obtained meal images and the obtained images other than the obtained meal images to the server apparatus upon obtaining the meal images and the images other than the meal images, which have not yet been transmitted to the server apparatus,
the server apparatus includes;
   a meal item storage in which meal items and image feature amounts of the corresponding meal items are stored in advance;
   a reception part configured to receive the meal images and the images other than the meal images from the user terminal;
   an image processing part that extracts the image feature amounts and the imaging dates and times of the meal image and the image other than the meal image received by the reception part;
   a meal item identifying part that identifies a meal item included in the meal image, based on a degree of matching between the image feature amount of the meal item stored in the meal item storage and the image feature amount of the meal image and the image other than the meal image extracted by the image processing part;
   a display part that displays a meal image analysis screen such that the meal image, for which the meal item has been identified, and the meal item, which has been identified in the meal image, are included in the meal image analysis screen, while not displaying the meal image other than the meal image for which the meal item has not been identified by the meal item identifying part; and
   a meal management information storage that stores meal management information based on the imaging date and time of the meal image and the meal item identified by the meal item identifying part.

The object and advantages of the embodiment will be realized and attained by means of the elements and combinations particularly pointed out in the claims. It is to be understood that both the foregoing general description and the following detailed description are exemplary and explanatory and are not restrictive of the invention.

### BRIEF DESCRIPTION OF THE DRAWINGS

FIG. 1 is a diagram showing a configuration example of a meal management system according to the present embodiment.

FIG. 2 is a diagram showing a hardware configuration example of a meal management system according to the present embodiment.

FIG. 3 is a diagram showing a software configuration example of a meal management system according to the present embodiment.

FIG. 4 is a diagram showing an example of data of a user management DB 107a according to the present embodiment.

FIG. 5 is a diagram showing an example of data of a meal item DB 107b according to the present embodiment.

FIG. 6 is a diagram showing an example of data of a meal management DB 107c according to the present embodiment.

FIG. 7 is a view showing an example of storing a meal image of the user terminal according to the embodiment.

FIG. 8 is a diagram showing an example of a meal image analysis screen of the meal management application according to the present embodiment.

FIG. 9 is a diagram showing an example of a meal image analysis screen of the meal management application according to the present embodiment.

FIG. 10 is a diagram showing an example of a meal image analysis screen of the meal management application according to the present embodiment.

FIG. 11 is a diagram showing an example of a meal image analysis screen of the meal management application according to the present embodiment.

FIG. 12 is a diagram showing an example of a meal image analysis screen of the meal management application according to the present embodiment.

FIG. 13 is a diagram showing an example of a meal management information screen of the meal management application according to the present embodiment.

FIG. 14 is a diagram showing an example of a meal management information screen of the meal management application according to the present embodiment.

FIG. 15 is a sequence diagram showing a meal registration process between a meal management server 10 and a user terminal 20 according to the present embodiment.

FIG. 16 is a diagram showing an example of data of a meal management DB 107c according to the present embodiment.

### DESCRIPTION OF EMBODIMENTS

In the following, embodiments will be described with reference to the accompanying drawings.

### <System Configuration>

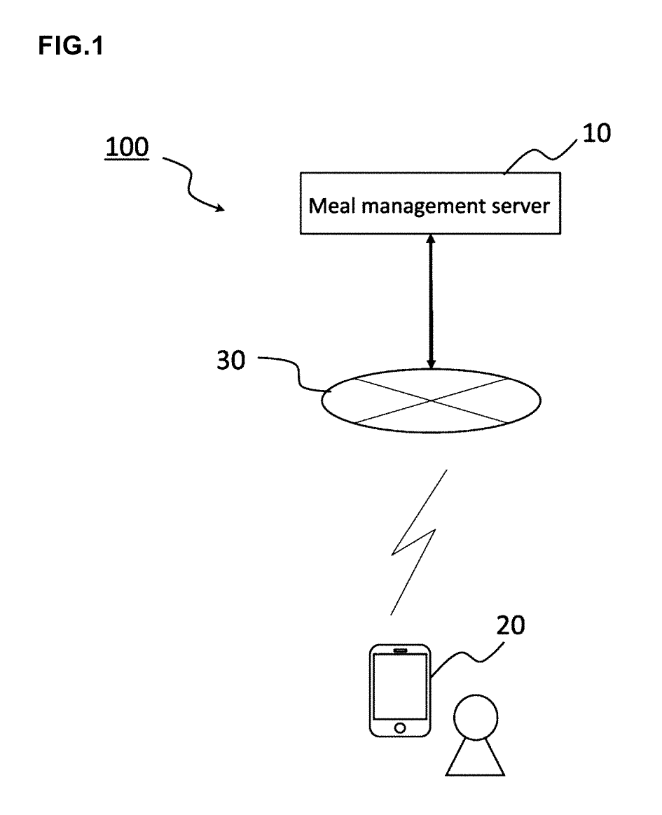

FIG. 1 is a diagram showing a configuration example of a meal management system according to the present embodiment. The meal management system 100 of FIG. 1 includes a meal management server 10 and a user terminal 20 which are connected via a network 30.

The meal management server 10 is a server apparatus that performs calorie calculation and nutrition management based on ingested meal items (meal menu). A user manages ingested meal to help the user's own health care and diet.

The user terminal 20 is a terminal device for performing a meal management, such as a smartphone, a tablet terminal, a PC, or the like. A meal management application (meal management application program) is installed in the user terminal 20 in advance. In addition, the user can take a meal image (meal photo) with a camera integrated in the user terminal 20 every time the user eats meal so that the stored meal images can be uploaded to the meal management server 10 by merely activating the meal management application at a convenient time. Since the uploaded meal images are automatically recognized collectively based on the image analysis by the meal management server 10, the meal items can be easily recorded afterward.

The network 30 is a communication network including wired and wireless. The network 30 includes, for example, Internet, a public line network, WiFi (registered trademark), and the like.

### <Hardware Configuration>

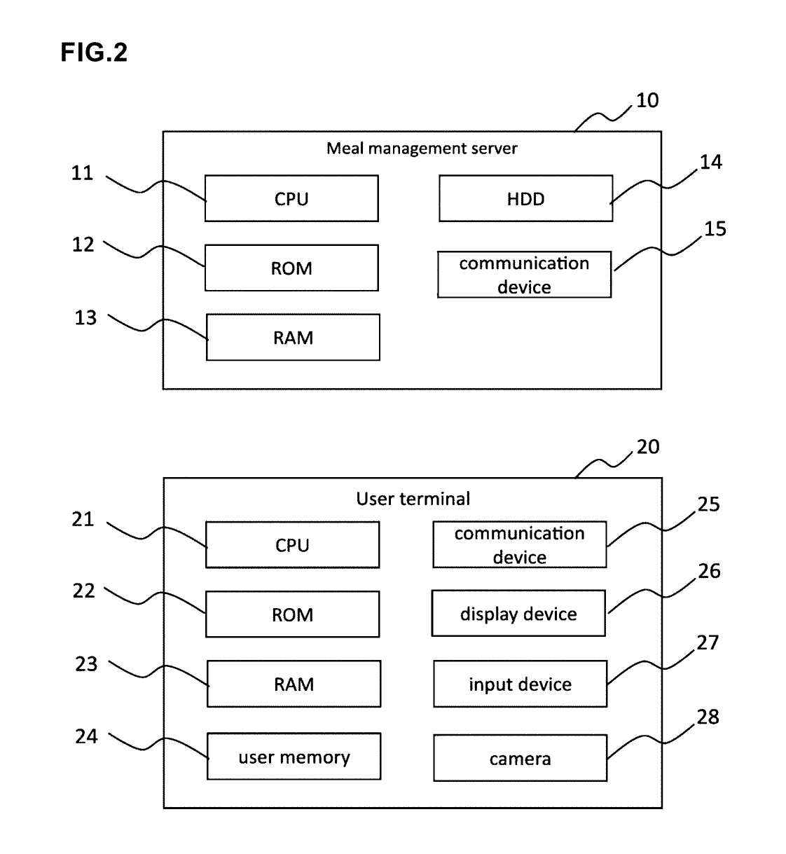

FIG. 2 is a diagram showing a hardware configuration example of the meal management system according to the present embodiment. FIG. 2, the meal management server 10 includes a CPU (Central Processing Unit) 11, a ROM (Read Only Memory) 12, a RAM (Random Access Memory) 13, an HDD (Hard Disk Drive) 14, and a communication device 15.

The CPU 11 executes various programs and performs arithmetic processing. The ROM 12 stores necessary programs and the like at the time of activation. The RAM 13 is a work area for temporarily storing processing by the CPU 11 and storing data. The HDD 14 stores various data and programs. The communication device 15 communicates with other devices via the network 30.

The user terminal 20 includes a CPU 21, a ROM 22, a RAM 23, a user memory 24, a communication device 25, a display device 26, an input device 27, and a camera 28.

The CPU 21 executes various kinds of programs and performs arithmetic processing. The ROM 22 stores necessary programs and the like at the time of activation. The RAM 23 is a work area for temporarily storing processing of the CPU 21 and storing data. The user memory 24 is an HDD or an SSD, stores various user data such as meal images and various programs such as a meal management application. The communication device 25 communicates with other devices via the network 30.

The display device 26 is a color display such as a liquid crystal display. The input device 27 is realized by unique operation keys, buttons, and the like. In addition, the input device 27 can be realized by a touch panel that can detect the tap coordinates (touch coordinates) on the display screen instead of the operation keys and buttons. In this case, the input operation is realized by the touch panel on the screen and a software key etc., controlled by the program.

The camera 28 is attached to the user terminal 20 and is an imaging device for picking up an image such as a meal image.

### <Software Configuration>

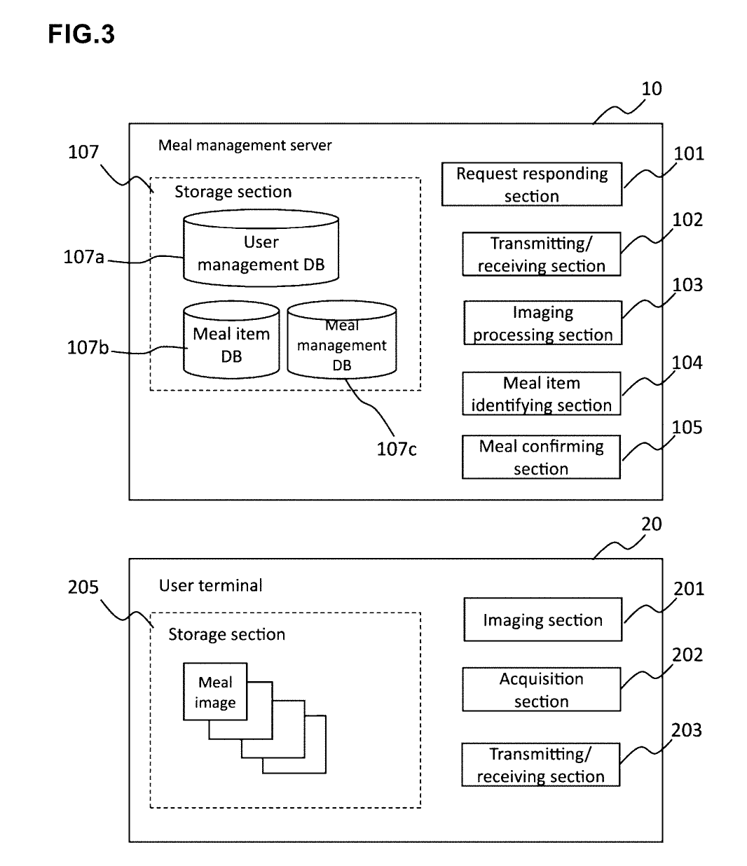

FIG. 3 is a diagram showing a software configuration example of the meal management system according to the present embodiment.

The meal management server 10 includes a request responding section 101, a transmission/reception section 102, an image processing section 103, a meal item identifying section 104, a meal item confirming section 105, and a storage section 107 as main functional sections.

The request responding section 101 transmits in response to a request from the user terminal 20, the latest imaging date and time among the imaging dates and times of the meal images stored in the meal management DB (Data Base) 107c.

The transmission/reception section 102 receives the meal image from the user terminal 20.

The image processing section 103 extracts the image feature amount and the imaging date and time of the meal image from the meal image received by the transmission/reception section 102.

The meal item identifying section 104 identifies, based on a degree of matching between the image feature amounts of the meal items registered in the meal item DB 107b and the image feature amount of the meal image extracted by the image processing section 103, the meal item included in the meal image.

The meal item confirming section 105 transmits, when a plurality of meal items is identified from one meal image by the meal item identifying section 104, the one meal image, the imaging date and time of the meal image, a plurality of meal items to be confirmed by the user to the user terminal 20.

The storage section 107 stores a user management DB 107a in which user information is registered in advance, a meal item DB 107b in which meal item information and image amounts of meal items and the like are registered in advance, and stores a meal management DB 107c in which the meal management information based on the meal items ingested by the user and the like are stored.

The user terminal 20 includes, as main functional units, an imaging section 201, an acquiring section 202, a transmitting/receiving section 203, and a storage section 205.

The imaging section 201 uses the camera 28 to capture a meal image including an imaging date and time.

The acquiring section 202 requests information of the latest imaging date and time to the meal management server 10 at the timing when the application program is activated, etc., and acquires, from the images captured and accumulated in the user terminal 20, the meal images whose imaging date and time is newer than the latest imaging date and time received from the meal management server 10.

The transmitting/receiving section 203 transmits the meal image acquired by the acquiring section 202 to the meal management server 10 as an untransmitted meal image.

The storage section 205 stores the meal image in the user memory 24 of the user terminal 20.

It is noted that each function section is realized by a computer program and a meal management application executed on hardware resources such as a CPU, a ROM, a RAM, etc. of a computer constituting the meal management server 10 and the user terminal 20. The functional sections may be replaced by “means”, “module”, “unit”, or “circuit”. Each DB in the storage section 107 in the meal management server 10 can also be arranged in an external storage device on the network 30.

### (Various Databases)

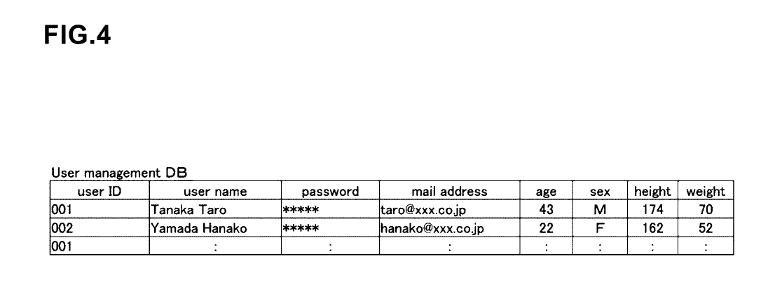

FIG. 4 is a diagram showing an example of data of the user management DB 107a according to the embodiment. The user management DB 107a is a database in which user information of a user is registered in advance, and includes data items such as, “user ID”, “password”, “mail address”, “age”, “sex”, “height”, “weight” and the like.

“User ID” indicates a unique identifier to be numbered for each user in terms of management. The user is the user of the user terminal 20 in the meal management system 100.

“Password” indicates a login/password of the meal management system 100.

“Mail address” indicates a mail address of the user.

“Age”, “sex”, “height”, and “weight” indicate age, sex, height, and weight as user attribute information.

It is noted that the user information can be inputted and registered in advance at the time of registering membership by the user.

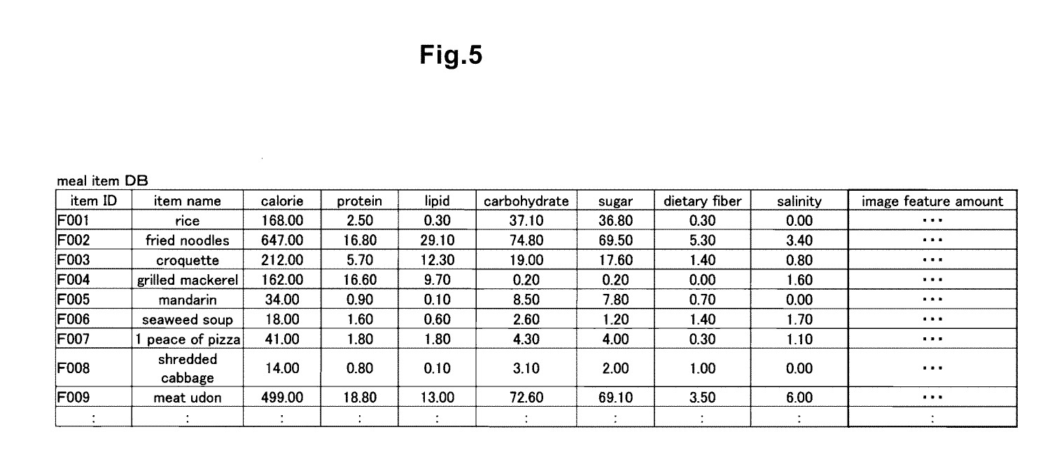

FIG. 5 is a diagram showing an example of data of the meal item DB 107b according to this embodiment. The meal item DB 107b is a database in which meal item information of various meals and image feature amounts of meal items are registered in advance, and includes data items such as, “item ID”, “item name”, “calorie”, “protein”, “lipid, “carbohydrate”, “sugar”, “meal fiber”, “salinity”, “image feature amount”, and the like.

“Item ID” indicates a unique identifier allocated to each meal item in terms of management.

“Item name” indicates a name of the meal item.

“Calorie” indicates calories of the meal item.

“Protein”, “lipid”, “carbohydrate”, “sugar”, “meal fiber” and “salinity” indicate the content of protein, lipid, carbohydrate, sugar, meal fiber, and salt in the meal item, respectively.

“Image feature amount” indicates feature amount data extracted from an image of a meal item. It is used when identifying the meal item in a meal image captured by the user based on comparison and similarity with “image feature amount” of the meal item. “Image feature amount” of the meal item has been extracted from a sample image of a large number of meal items in advance for the purpose of improving precision, but after a start of operation, more numerous feature amounts are automatically extracted from the user's meal images, such that the feature of the image of the meal item may be learned with higher precision as “image feature amount” of the meal item (deep learning).

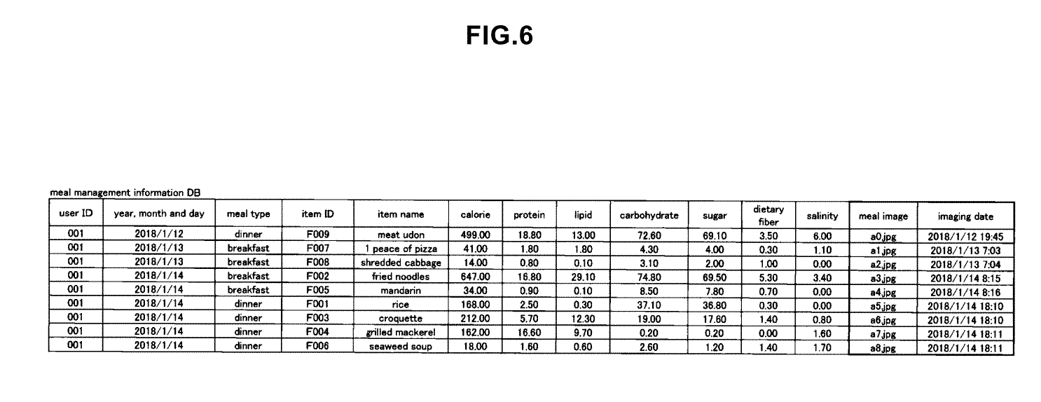

FIG. 6 is a diagram showing an example of data of the meal management DB 107c according to the present embodiment. The meal management DB 107c is a database for managing information on meals ingested by the user and includes, for example, “user ID”, “year, month, day”, “meal type”, “item ID”, “item name”, “calorie”, “protein”, “lipid”, “carbohydrate”, “sugar”, “meal fiber”, “salinity” ,“meal image”, “imaging date and time”, and the like.

“User ID” indicates the user ID of the user who ate the meal.

“Year/Month/Day” indicates the year, the month and the day when the user took the meal.

“Meal type” indicates whether the meal includes breakfast, lunch, dinner, or snack.

“Item ID” indicates the item ID of the item that meal the user ate.

“Item name” indicates the item name of the meal that the user ate.

“Calories”, “protein”, “lipid”, “carbohydrate”, “sugar”, “meal fiber” and “salinity” are “calories”, “protein”, “lipid”, “carbohydrate”, “sugar”, “meal fiber” and “salinity” included in the meal that the user ate.

“Meal image” indicates meal image data imaged by the user.

“Imaging date and time” indicates imaging date and time of “meal image”. The imaging date and time can be obtained from Exif information that an image generally includes.

### <Application Example>

### (Example of Storing a Meal Image)

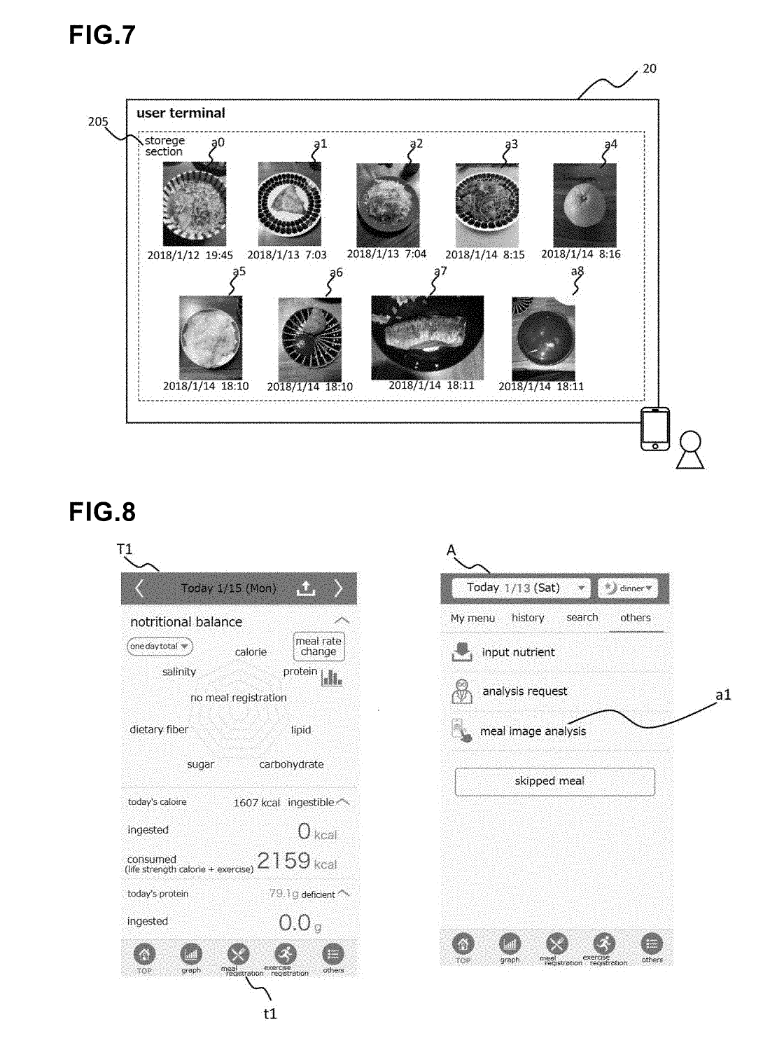

FIG. 7 is a view storing an example of saving a meal image by the user terminal according to the present embodiment.

When eating, the user uses the camera 28 of the user terminal 20 to capture a meal image (picture). The captured meal images a0 to a8 are stored in the user memory 24 of the user terminal 20. In meal management system 100, the user need not transmit the meal image to the meal management server 10 every time when the user eats the meal. The user takes only the meal image every meal such that the meal image s are accumulated.

The user can transmit the meal images to the meal management server 10 at a convenient time. Specifically, when the user activates the meal management application of the user terminal 20, the meal management application accesses the meal management server 10 such that, when it is assumed that the meal image a0 was transmitted on 1/12, among the meal images a0 to a8, the untransmitted meal images a1 to a8 taken and stored between 1/13 and 14 are automatically uploaded to the meal management server 10.

It is noted that, in addition to automatically uploading the meal images when the user activates the meal management application, it is also possible to manually upload the meal images (meal pictures) to the meal management server10 by selecting a meal images the user wants to upload from the meal management application and performing a transmission operation.

In addition, the user can also take images (picture) other than a meal image using the camera 28. The image is stored in the user memory 24 of the user terminal 20 like the meal image.

### (Meal Image Analysis)

FIG. 8 is a diagram showing an example of a meal image analysis screen of the meal management application according to the present embodiment.

When the user activates the meal management application of the user terminal 20, the top screen T1 of the application is displayed. In addition, the meal images a1 to a8 that were captured and accumulated as described above are automatically uploaded to the meal management server 10.

When “meal registration” t1 is operated on the top screen T1, a transition is made to a meal registration screen A. The meal registration screen is used for inputting information such as the meal item the user has eaten, the date, the meal type, the amount of meal eaten etc. There are several input methods, but here, when “meal image analysis” a1 is operated to use the “meal image analysis” function of the meal management application, the screen changes to the meal analysis result screen B.

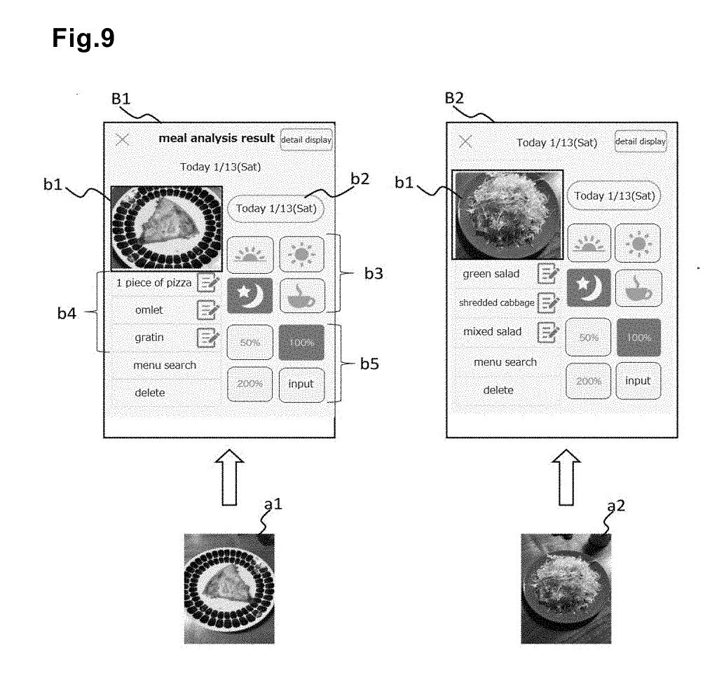

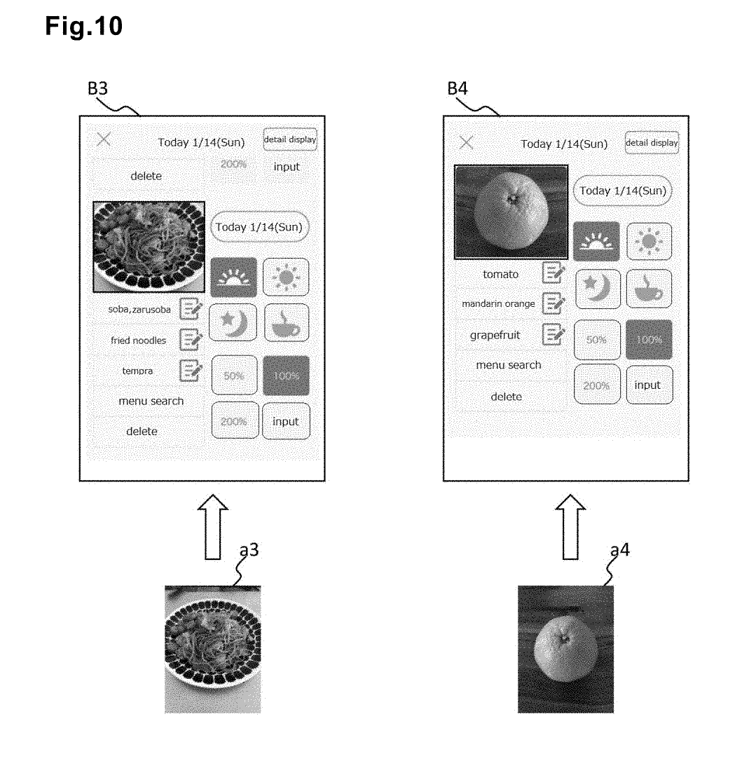

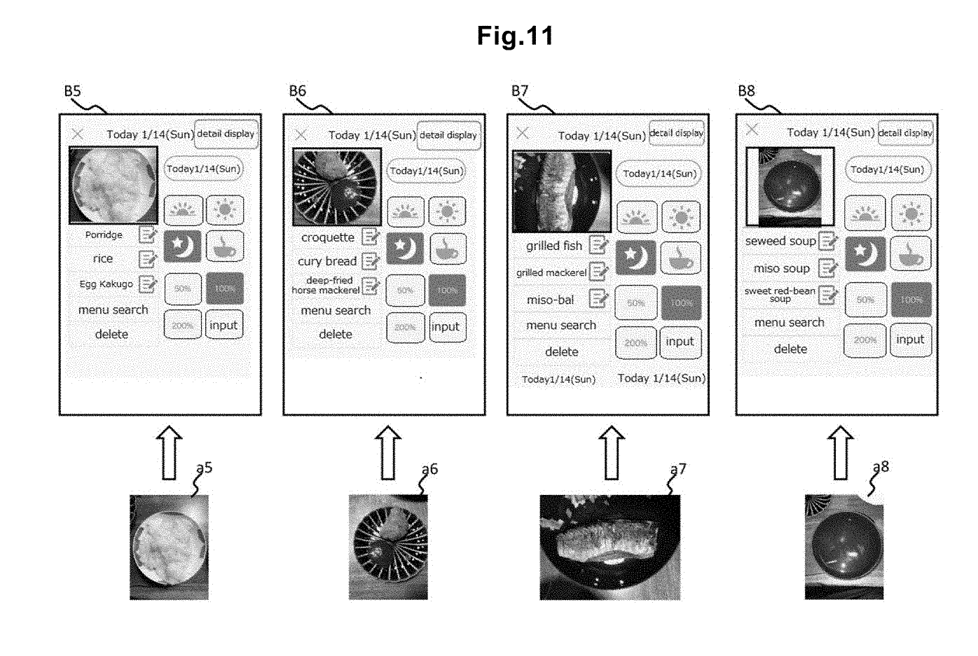

FIG. 9 to FIG. 11 are diagrams showing an example of the meal image analysis screen of the meal management application according to the present embodiment. On the meal image analysis screen, the meal item identified based on the uploaded meal image is displayed. The meal image analysis screens B1 to B8 include a meal image b1, a date b2, a meal type selection field b3, a meal item selection field b4, and an ingestion rate selection field b5.

The meal image b1 indicates a meal image to be subject to a meal image analysis.

Date b2 indicates the day when the user ate the meal. It is automatically extracted based on the imaging date and time of the meal image.

The meal type selection field b3 is a field for selecting and inputting the meal type of the meal. By default, either breakfast, lunch, dinner, or snack is automatically extracted based on the imaging date and time of the meal image, but it is also possible for the user to selectively modify it. For example, in the case of a breakfast in a late time period, since it can be determined that lunch is based on the imaging date and time of the meal image, the user corrects the type from lunch to breakfast.

The meal item selection field b4 is a field in which the user selects and inputs the correct meal item from a plurality of meal items. When a plurality of meal items is identified from the meal image to be analyzed by the meal image analysis, the user selects and inputs one correct meal item. For example, even if the correct meal item is pizza, it may be analyzed as other meal items such as omelets and gratin due to imaging conditions (color, shape, angle, etc.) of the meal image. In this case, the user selects, among them, the pizza as a correct answer which is captured in the meal image. It is noted that, when there is no correct answer in the meal item selection field b4, the correct answer can be input from “menu search”. In addition, when only one meal item is identified from the meal image to be analyzed by the meal image analysis, it is unnecessary to select a meal item.

The ingestion rate selection field b5 is a field for selecting and inputting the ingestion rate of the meal item of the meal in order to identify the amount and percentage of the meal item eaten by the user. The default is set to 100%, but it is also possible to select or correct the percentage of the meal item eaten by the user. For example, if the user only eats half of the meal item, the percentage is modified to 50%.

As shown in FIG. 9 to FIG11, among the results of the meal analysis for the meal images a1 to a8, the correct meal items selected and inputted by the user are as follows.

Meal image a1: 1/13 (Saturday) Dinner 1 piece of pizza (meal item)

Meal image a2: 1/13 (Saturday) Dinner shredded cabbage (meal item)

Meal image a3: 1/14 (Sunday) Breakfast fried noodles (meal items)

Meal image a4: 1/14 (Sunday) Breakfast mandarin (meal item)

Meal image a5: 1/14 (Sunday) Dinner rice (meal item)

Meal image a6: 1/14 (Sunday) Dinner croquette (meal item)

Meal image a7: 1/14 (Sunday) Dinner grilled mackerel (meal item)

Meal image a8: 1/14 (Sunday) Dinner seaweed soup (meal item)

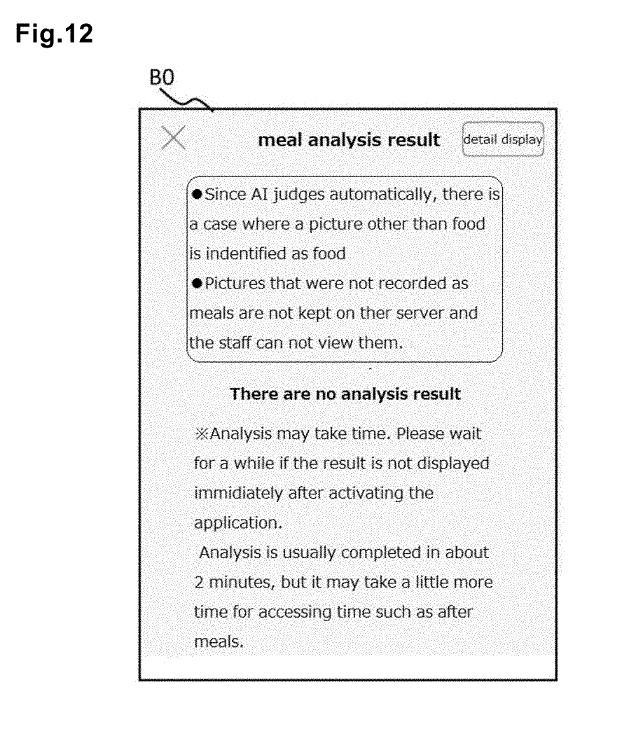

It is noted that, when there is no target meal image, the meal item is not displayed on the meal image analysis screen (B0 in FIG. 12). Also, even when an image (picture) other than the meal image is to be analyzed by the meal image analysis, the meal item is not extracted from the image, and thus the meal item is not displayed on the meal analysis result screen.

### (Meal Management Information)

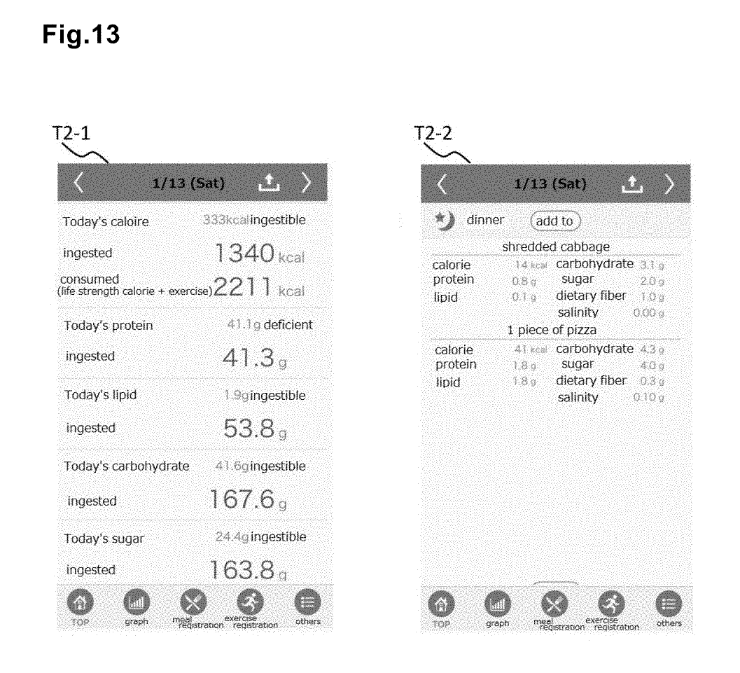

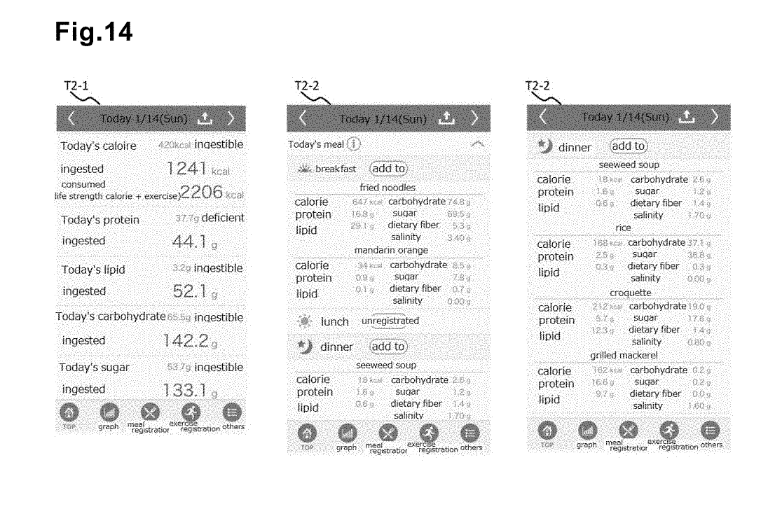

FIG. 13 and FIG. 14 are diagrams showing an example of the meal management information screen of the meal management application according to the present embodiment. When the meal registration (FIG. 9 to FIG. 11) is performed, information is reflected in the meal management information on the meal management information screen T2-1. The meal management information of the meal management information screen T2-1 includes “calorie”, “meal”, “protein”, “lipid”, “carbohydrate”, “sugar”, “meal fiber”, “salinity”, etc., of the meal, which the user ate at the imaging date and time of the meal image, are displayed. Also, on the meal management information screen T2-2, a breakdown of the meal item of the meal and the meal management information at the day of the photographing date and time of the meal image is displayed for each meal type. Specifically, on each day of the imaging date and time of the meal images a1 to a8, the meal management information of the meal item based on the meal images a1 to a8 and its breakdown are displayed for each meal type.

For example, in the case of dinner of 1/13 (Saturday), one piece of shredded cabbage, one piece of pizza, and each “calorie”, “protein”, “lipid”, “carbohydrate”, “sugar”, “meal fiber”, “salinity ” thereof are displayed.

Also, in the case of breakfast of 1/14 (Saturday), fried noodles, mandarin, and in the case of dinner of 1/14 (Saturday), seaweed soup, rice cake, croquette, baked mackerel and each “calorie”, “protein”, “lipid”, “carbohydrate”, “sugar”, “meal fiber”, “salinity ” thereof are displayed.

### <Meal Registration Processing>

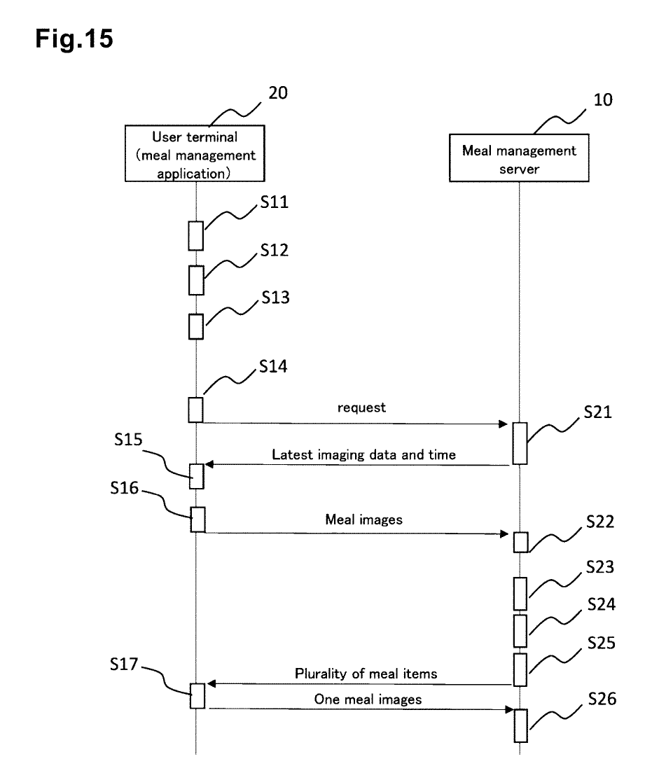

FIG. 15 is a sequence diagram showing a meal registration process between the meal management server 10 and the user terminal 20 according to the present embodiment.

S11˜S13: First, at meal time, the meal image (picture) of the meal that the user eats is taken with the camera 28 of the user terminal 20. The user takes only the meal image every meal to accumulate the meal images. The imaging section 201 of the user terminal 20 takes a meal image including the imaging date and time, and the storage section 205 stores the meal image in the user memory 24.

S14: The user activates the meal management application at an arbitrary timing convenient for the user. The acquiring section 202 of the user terminal 20 requests the meal management server 10 for information on the latest imaging date and time at the timing when the meal management application is activated. The request includes the user ID of the meal management application.

S21: In response to the request from the user terminal 20, the request responding section 101 of the meal management server 10 transmits (responds) the latest imaging date and time among the imaging dates and times of the meal images stored in the meal management DB 107c.

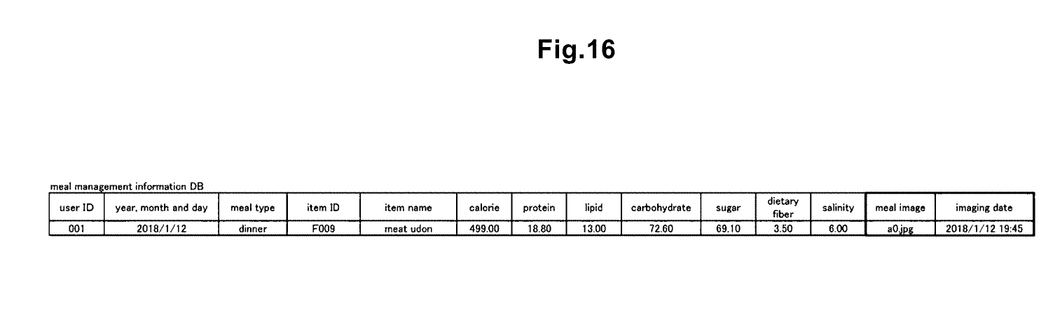

FIG. 16 is a diagram showing an example of data of the meal management DB 107c according to this embodiment. For example, in the case of the meal management DB 107c of FIG. 15, the latest imaging date and time “2018/1/12 19: 45” among the imaging dates and times of the meal images is transmitted.

S15: The acquiring section 202 of the user terminal 20 acquires all the meal images of the imaging dates and times that are newer than the received latest imaging date and time from the user memory 24. As a result, all the undelivered meal images (that is, the meal images taken and accumulated during a period from the last transmission to the current time) are transmitted to the meal management server10.

In the case of FIG. 7, the meal images a1 to a8 of the imaging dates and times newer than the received latest imaging date and time “2018/1/12 19: 45” are acquired from the user memory 24. The meal image a0 is not acquired because the imaging date and time is not newer than the latest imaging date and time “2018/1/12 19: 45”, that is, the meal image a0 has been already transmitted.

S16: The transmitting/receiving section203 of the user terminal 20 transmits all the meal images acquired in S15 to the meal management server 10.

S22: The transmitting/receiving section 102 of the meal management server 10 receives the meal images from the user terminal 20.

S23: The image processing section 103 of the meal management server 10 extracts the image feature amount and the imaging dates and times of the meal images of the received meal images. The method of extracting the image feature amount of the meal image may be one of any conventional method, and the imaging date and time can be acquired from the Exif information of the meal image.

S24: The meal item identifying section 104 of the meal management server 10 identifies the meal item whose degree of matching between the image feature amount of the meal item registered in the meal item DB 107b and the image feature amount of the meal image extracted in S23 is equal to or greater than a predetermined value (that is, identifies a meal item whose image feature amount is similar to or greater than a predetermined threshold value), such that the meal item included in the meal image is identified.

S25: When a plurality of meal items is identified from one meal image in S24, the meal item confirming section 105 of the meal management server 10 transmits the meal image, the imaging date and time of the meal image, and the plurality of the meal items to be confirmed by the user to the user terminal 20. As a result, the meal image, the imaging date and time of the meal image, and the plurality of meal items to be confirmed by the user are displayed on the meal management application.

For example, in the case of the meal image a1 in FIG. 9, a plurality of meal items “one piece of pizza”, “omelette”, and “gratin” are identified from the meal image a1. In the meal image analysis screen B1 of the meal management application, the meal image a1, 1/13 of the date b2 corresponding to the imaging date and time of the meal image, dinner in the meal type selection field b3, and the meal items “one piece of pizza”, “omelette” and “gratin” in the meal item selection field b4 to be confirmed by the user are displayed.

S17: When the user inputs and confirms a correct one meal item based on the imaging date and time of the meal image and the meal image, the input and selected meal item is transmitted to the meal management server 10.

In the case of the meal image a 1 of FIG. 9, one diet item “one piece of pizza” is selected and input.

S26: The storage section 107 of the meal management server 10 stores the meal management information, the meal image, the imaging date and time of the meal image based on the imaging date and time of the meal image and the meal item identified at S17 in the meal item database 107b.

In the case of the meal image a.1 of FIG. 9, as the meal management information based on the imaging date and time “2018/1/13 7:03” of the meal image and the meal item “one piece of pizza”, the year, month, and day(2018/1/13), meal type(breakfast) “item ID” corresponding to “one piece of pizza”, “item name”, “calorie”, “protein”, “lipid”, “carbohydrate”, “sugar”, “meal fiber” , “salinity”, in addition, the meal image (a1.jpg) and the imaging date and time (2018/1/13 7: 03) of the meal image in the meal item DB 107b.

It is noted that in S15, the acquiring section 202 of the user terminal 20 may acquire not only the meal images of the imaging dates and times newer than the received latest imaging date and time as the target meal image to be acquired from the user memory 24 but also the meal image for which the following condition is met.
Meal images whose imaging dates and times are within 72 hours from the current date and time, for example. This is because users are recommended to update as frequently as possible (upload) to exclude too old meal images. This also leads to the continuation of the meal management.
The user terminal 20 is in a Wi-Fi (registered trademark) network environment and an image capacity of the meal image is 5 MB or less. This is to reduce the burden on the image transmission of the user terminal 20.

### <Summary>

Conventionally, in the case of a meal management, a user needs to take the trouble of having to record the date (year, month, day), the meal type, and the meal item every time the meal is taken. If the user forgets to record, it is also possible to collectively record while remembering past meals. However, it is difficult to accurately recall all the memories of the meal items eaten afterwards, so it is highly likely that meal management itself will not continue for a long time due to inability to manage accurate meals, for example.

According to the present embodiment, the user can take a meal image (meal picture) with the camera attached to the user terminal 20 every time the user eats the meal, and accumulate them so that the user can upload the meal images to the meal management server 10 by just activating the meal management application at a convenient time. The meal items are automatically and collectively recognized from the uploaded meal images by the image analysis of the meal management server 10.

That is, the user may only take and store each meal image that he ate every meal. After that, the user simply activates the meal management application at a convenient time, so that, on the meal management server 10, the date (year, month, day), the meal type, the meal item, the nutritional ingredient of each meal are automatically registered. Therefore, the meal registration on the meal management application is simplified, and thus the user can easily continue the meal management and diet without feeling the burden of recording.

As described above, the meal management system 100 according to the present embodiment makes it unnecessary for every meal to be recorded which makes it possible in the meal management.

It is to be noted that, although the present invention has been described with reference to specific embodiments according to preferred embodiments of the present invention, it is to be understood that these embodiments may be modified without departing from the broader spirit and scope of the invention as defined in the appended claims. It is obvious that various modifications and changes can be made to the examples. That is, the present invention should not be construed as being limited by the details of specific examples and the attached drawings.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present inventions have been described in detail, it should be understood that the various changes, substitutions, and alterations could be made hereto without departing from the spirit and scope of the invention.

What is claimed is:

1. A meal management system for managing a meal of a user, the meal management system including a user terminal in which an application program is to be executed and a server apparatus, wherein
the application program causes a computer to:
obtain a meal image and an image other than the meal image, the meal image including information of date and time when the meal image was captured;
store the meal images and the images other than the meal images in an image storage;
obtain the meal images and the images other than the meal images, which have not been transmitted to the server apparatus, from the image storage at a timing when the application program is activated; and
transmit all the obtained meal images and the obtained images other than the obtained meal images to the server apparatus upon obtaining the meal images and the images other than the meal images, which have not yet been transmitted to the server apparatus,
the server apparatus includes;
a meal item storage in which meal items and image feature amounts of the corresponding meal items are stored in advance;
a reception part configured to receive the meal images and the images other than the meal images from the user terminal;
an image processing part that extracts the image feature amounts and the imaging dates and times of the meal image and the image other than the meal image received by the reception part;
a meal item identifying part that identifies a meal item included in the meal image, based on a degree of matching between the image feature amount of the meal item stored in the meal item storage and the image feature amount of the meal image and the image other than the meal image extracted by the image processing part;
a display part that displays a meal image analysis screen such that the meal image, for which the meal item has been identified, and the meal item, which has been identified in the meal image, are included in the meal image analysis screen, while not displaying the meal image other than the meal image for which the meal item has not been identified by the meal item identifying part; and
a meal management information storage that stores meal management information based on the imaging date and time of the meal image and the meal item identified by the meal item identifying part.

2. The meal management system according to claim 1, wherein
the computer, when obtaining the meal image and the image other than the meal image, is caused to transmit a request for information on the latest imaging date and time to the server apparatus, and obtain, from the image storage, the meal image whose imaging date and time are newer than the latest imaging date and time received from the server apparatus, and
the server apparatus further includes a request responding part transmits the latest imaging date and time among the imaging dates and times of the meal images stored in the meal management information storage in response to the request.

3. The meal management system according to claim 1, wherein
the server apparatus further includes a meal item confirming part that transmits, in response to a plurality of meal items being identified from the one meal image by the meal item identifying part, the imaging date and time of the one meal image, the meal image, and the plurality of meal items, which are to be confirmed by the user, to the user terminal, and
the meal management information storage stores, in response to one of the plurality of meal items being confirmed to be inputted from the user terminal, the meal management information based on the imaging date and time of the meal image and the confirmed one of the meal items.
